(19) United States
(12) Patent Application Publication
(10) Pub. No.: US 2024/0151711 A1
(43) Pub. Date: May 9, 2024

(54) ADJUVANT FOR VACCINE DEVELOPMENT

(71) Applicant: Haifa SHEN, Woodcliff Lake, NJ (US)

(72) Inventor: Haifa SHEN, Woodcliff Lake, NJ (US)

(21) Appl. No.: 18/549,868

(22) PCT Filed: Mar. 14, 2022
(86) PCT No.: PCT/IB2022/052241

§ 371 Date: Sep. 8, 2023

Related U.S. Application Data
(60) Provisional application No. 63/160,852, filed on Mar. 14, 2021.

(51) Int. Cl. G01N 33/50

(52) U.S. Cl. CPC G01N 33/5052; G01N 33/5055; G01N 2333/525; G01N 2333/565

(57) ABSTRACT

The present invention provides a cell-based method for identification of an adjuvant and adjuvant combinations and a composition of a vaccine that includes the adjuvant and adjuvant combinations. The method comprises the steps: using an adjuvant or adjuvant combination to treat at least one type of antigen-presenting cells and measuring amount of at least one cytokine produced by the antigen-presenting cells.

### CROSS-REFERENCE TO RELATED APPLICATIONS

This application claims the priority of an USA provisional application with the application No. 63/160,852, filed on 14 Mar. 2021, which is incorporated herein by reference in its entirety, including the description, claims and drawings.

### FIELD OF THE INVENTION

The present invention relates methods on identification of adjuvants and adjuvant combinations for vaccine development. The present invention also relates composition of adjuvants and adjuvant combinations identified based on these methods. Adjuvant is an essential part of a vaccine for prevention and treatment of diseases, and potency of the adjuvant determines effectiveness of the vaccine. The current invention describes cell-based methods to identify adjuvants and adjuvant combinations. The current invention also describes preparation of vaccines using the identified adjuvants or adjuvant combinations.

### DESCRIPTION OF PRIOR ART

Innate immune recognition of cancer is a critical step for spontaneous tumor-specific T cell priming and subsequent T cell infiltration (1). Antigen-presenting cells, mainly dendritic cells (DCs), capture tumor-derived antigens and danger signal molecules, and process an antigen to form antigen epitope-major histocompatibility complex (MHC) that is then presented to T cells and activates these cells together with co-stimulation signals on DC cell surface (2). Stimuli such as pathogen-associated molecular patterns (PAMPs) from invading microbes or danger-associated molecular patterns (DAMPs) released from dying tumor cells can bind to and activate pattern recognition receptors (PRRs) on DCs. The process in turn promotes DC activation and primes appropriate T cell responses, thereby bridging innate and adaptive immunity (1, 3). Effective T cell priming requires not only specific TCR-antigen recognition and co-stimulation signals, but also T cell-activating cytokines from DCs (4). Type I interferons and inflammatory cytokines are critical both for DC maturation and for effective T cell priming (5). These immune-activating cytokines can be induced from innate immune receptor activation by tumor-derived ligands or artificially administrated adjuvants. Indeed, intra-tumoral administration of the Toll-like receptor 9 (TLR9) ligand CpG oligonucleotide (CpG) or stimulator of interferon genes (STING) agonist 5,6-dimethylxanthenone-4-acetic acid (DMXAA) can elicit strong antitumor immunity by promoting T cell priming and tumor killing while relieving immune suppression (6, 7).

Therapeutic cancer vaccines can effectively boost cancer immune recognition and promote antitumor immunity. To facilitate DC maturation and effective T cell priming, vaccines often contain soluble or particulate adjuvants that stimulate innate immunity, promote antigen presentation, and induce co-stimulation signals and helper cytokines (8). Many types of PAMPs including TLR ligands, NOD-like receptor ligands, RIG-I-like receptor ligands have been evaluated for their antitumor potency (9). Some have been formulated in nanoparticles and microparticles (3). Interestingly, certain nanoparticles and microparticles also have adjuvant-like properties. For an example, nanoporous silicon microparticles (μ-particles) have been shown to stimulate a mild but significant level of IFN-I response in DCs by activating TRIF- and MAVS-dependent pathways, and exhibit prolonged early endosome localization that promotes antigen processing and cross-presentation (10). Another example is mRNA nanoparticles composed of antigen-expressing mRNA molecules packaged in certain forms of lipid-based shells. Theyare capable of mildly stimulating TLR7/8 signaling (11). But not all particles can be applied to prepare cancer vaccines that rely on Th1-biased immune response. Aluminum salt (alum) is a particulate adjuvant that activates the inflammasomes (12, 13), and is one of the most common particulate adjuvants for human vaccines; however, its application in therapeutic cancer vaccine development has been unsuccessful so far, mainly due to its preference to stimulate a Th2-biased immune responses (14).

Thus, it is essential to identify potent adjuvants and their compatible formulations in order to develop an effective vaccine.

### SUMMARY OF THE INVENTION

The present disclosure is directed to methods that are applied to identify adjuvants or adjuvant combinations capable of stimulating antigen-presenting cells.

In an embodiment, adjuvant activity is greatly enhanced when an adjuvant molecule is packaged into a nanoparticle or microparticle. In an exemplary embodiment, adjuvant molecules are packaged into nanopores in a microparticle, and the resulting particulate combination can strongly stimulate antigen-presenting cells to produce interferon-β (IFN-β) and tumor necrosis factor-α (TNF-α).

In another exemplary embodiment, adjuvant molecules are packaged together with mRNA molecules in lipid nanoparticles, and the resulting particulate mRNA vaccine promotes antigen-presenting cells to produce IFN-β and TNF-α.

The present disclosure is also directed to compositions of prophylactic and therapeutic vaccines. The compositions disclosed herein are comprised of at least one form of nanoparticle or microparticle, at least one adjuvant molecule, and at least one antigen or antigen source.

In an embodiment, the nanoparticle or microparticle is composed of porous silicon or porous silica. In an exemplary embodiment, at least one adjuvant molecule and one antigen molecule are packaged together with a porous silicon particle to form a particulate vaccine. In another embodiment, the nanoparticle is composed of mRNA molecules and lipids. In an exemplary embodiment, the mRNA molecule encodes an antigen protein or peptide, and one of the lipid molecules has the activity to stimulate antigen-presenting cells. In another exemplary embodiment, the antigen-encoding mRNA molecule also serves as an adjuvant to stimulate antigen-presenting cells.

So, one of the aspects, the invention provides a method for identification of adjuvants and adjuvant combinations, comprises the steps: using at least one type of hydrophilic or hydrophobic molecule to treat antigen-presenting cells and measuring amount of cytokine expression by the antigen-presenting cells.

In some embodiments, at least one cytokine has the property to stimulate antigen-presenting cells.

In some embodiments, the antigen-presenting cell is a dendritic cell, a macrophage, or a B lymphocyte. In some embodiments, the dendritic cell is derived from bone marrow cells. In some embodiments, the dendritic cell is isolated from peripheral blood or a tissue. In some embodiments, the dendritic cell is an immortalized cell line.

In some embodiments, the hydrophilic or hydrophobic molecule is capable of stimulating expression of a type I interferon or an inflammatory cytokine. In some embodiments, the hydrophilic or hydrophobic molecule is a Toll-like receptor ligand or agonist. In some embodiments, the hydrophilic or hydrophobic molecule is a STING agonist. In some embodiments, the hydrophilic or hydrophobic molecule is a nucleotide analogue. In some embodiments, the hydrophilic or hydrophobic molecule is selected from a compound library based on its cytokine-stimulating property. In some embodiments, the hydrophilic or hydrophobic molecule is an mRNA molecule.

In some embodiments, the cytokine can stimulate maturation of the antigen-presenting cells. In some embodiments, the cytokine is interferon-beta (IFN-β), In some embodiments, the cytokine is tumor necrosis factor-alpha (TNF-α).

In some embodiments, the hydrophilic or hydrophobic molecule can be packaged into a nanometer-size or micrometer-size particle. In some embodiments, the particle is a porous silicon particle, a porous silica particle, or a lipid nanoparticle. In some embodiments, the hydrophilic or hydrophobic molecule packaged in a particle can stimulate cytokine expression in antigen-presenting cells. In some embodiments, the hydrophilic or hydrophobic molecule packaged in a particle has an equal or greater activity in stimulating cytokine expression in antigen-presenting cells compared to its free form. In some embodiments, the hydrophilic or hydrophobic molecule synergizes with other components in the particle in stimulating cytokine expression in antigen-presenting cells. In some embodiments, the hydrophilic or hydrophobic molecule in the particle has the capacity to promote antigen processing and presentation in antigen-presenting cells.

In the second aspect, the invention provides a composition for the formulation of a vaccine, comprising: at least one antigen or antigen source; at least one hydrophilic or hydrophobic adjuvant; and at least one formulation to combine the adjuvant and antigen, wherein the at least one adjuvant is selected based on a cell-based assay.

In some embodiments, the antigen is a peptide, a protein, a collection of cells, or a disease tissue. The antigen source is a nucleic acid that encodes a protein, a peptide, or a group of peptides.

In some embodiments, the adjuvant or adjuvant combination is packaged together with the antigen or antigen source to form a vaccine. The adjuvant could be a CpG oligonucleotide (CpG), a cyclic GMP-AMP (cGAMP), a single strand RNA, monophosphoryl lipid A (MPLA), polyinosinic and polycytidylic acid (polyI:C), R848, imiquimod, or a multi-pattern recognition receptor ligand. The adjuvant combination could be selected from CpG, cGAMP, single strand RNA, MPLA, polyI:C, R848, imiquimod, or a multi-pattern recognition receptor ligand. The adjuvant combination could be CpG and cGAMP, CpG and MPLA, cGAMP and MPLA, cGAMP and R848, cGAMP and MPLA, cGAMP and R848.

In some embodiments, the vaccine is in the form of a nanometer-size or micrometer-size particle. The particulate vaccine is in the form of a liposome, a hydrogel, a polymeric nanoparticle, a silicon oxide nanoparticle, or a porous silicon particle.

In some embodiments, the adjuvant combination is an adjuvant and another component of the vaccine particle. In some embodiments, the other component is a porous silicon particle.

In some embodiments, the adjuvant combination is a group of adjuvants and another component of the vaccine particle. In some embodiments, the adjuvant combination is CpG, cGAMP, and porous silicon particle.

In some embodiments, the vaccine is an mRNA nanoparticle. The nanoparticle is composed of mRNA and a lipid shell. In some embodiments, at least one lipid component has adjuvant activity. In some embodiments, a least one lipid component is a STING agonist. The mRNA molecule and a lipid component synergize stimulation of cytokine production in antigen-presenting cells.

In the third aspect, the invention provided a new use of an adjuvant; or adjuvant combination for preparing a formulation of a vaccine. In some embodiments, the adjuvant is a CpG oligonucleotide (CpG), a cyclic GMP-AMP (cGAMP), a single strand RNA, monophosphoryl lipid A (MPLA), polyinosinic and polycytidylic acid (polyI:C), R848, imiquimod, or a multi-pattern recognition receptor ligand.

In some embodiments, the adjuvant combination is selected from CpG, cGAMP, single strand RNA, MPLA, polyI:C, R848, imiquimod, or a multi-pattern recognition receptor ligand. The adjuvant combination is CpG and cGAMP, CpG and MPLA, cGAMP and MPLA, cGAMP and R848, cGAMP and MPLA, cGAMP and R848, or MPLA and R848.

In some embodiments, the adjuvant combination is an adjuvant molecule and a particulate component of the vaccine. The particulate components is a porous silicon particle, a porous silica particle, or a lipid nanoparticle. In some embodiments, the lipid nanoparticle contains a STING agonist. In some embodiments, the adjuvant combination is CpG and porous silicon particle.

In some embodiments, the adjuvant combination is a group of adjuvants and a particulate component of the vaccine. The group of adjuvants are selected from at least one of these groups: CpG and cGAMP, CpG and MPLA, cGAMP and MPLA, cGAMP and R848, cGAMP and MPLA, cGAMP and R848, or MPLA and R848.

In some embodiments, the formulation of a vaccine contains an antigen or antigen source. In some embodiments, the antigen is a peptide, a protein, a collection of cells, or a disease tissue. The antigen source is a nucleic acid that encodes a protein, a peptide, or a group of peptides.

In some embodiments, the formulation disclosed above is used in the manufacture of a medicament for preventing, diagnosing, treating, or ameliorating, in a mammalian subject. In some embodiments, the mammalian subject is a human, a non-human primate, a companion animal, an exotic species, livestock, or feedstock animal.

### BRIEF DESCRIPTION OF THE DRAWINGS

A full understanding of the invention can be gained from the following description of the preferred embodiments when read in conjunction with the accompanying drawings in which:

FIG. 1 displays expression of Toll-like receptors (TLR3, TLR4, TLR7, TLR9) and STING in GM-CSF/IL-4-induced bone marrow-derived dendritic cells (GM-CSF/IL-4-BMDC), Flt3 ligand (Flt3L)-induced CD8⁺DCs (Flt3L-CD8⁺ DC), Flt3L-induced plasmacytoid DCs (Flt3L-pDC), splenic CD8⁺ DCs, splenic pDCs, and immortalized DC2.4 cells. Protein expression levels were analyzed with flow cytometry after cells were permeablized and stained with antibodies. Dash lines represent unstained DCs, and solid curves display stained cells. Individual proteins are listed on top of the panels.

FIG. 2 shows IFN-β expression level in culture media of GM-CSF/IL-4-BMDCs after cells were incubated with either single agents or their combinations for 24 hours. Concentrations of the reagents are: 2.5 μg/mL CpG oligonucleotide (CpG), 1.25 μg/mL 2′3′-cyclic GMP-AMP (cGAMP), 0.5 μg/mL monophosphoryl lipid A(MPLA), 0.5 μg/mL polyinosinic and polycytidylic acid (polyI:C), 0.5 μg/mL Resiquimod (R848). Phosphate buffer saline (PBS) served as a negative control. The result indicates that combinations of CpG+cGAMP, CpG+MPLA, cGAMP+MPLA, cGAMP+R848 and MPLA+R848 can synergistically stimulate IFN-β expression in BMDCs.

FIG. 3 shows TNF-α expression in culture media of GM-CSF/IL-4-BMDCs after cells were incubated with single agents or their combinations for 24 hours. Concentrations of the reagents are: 2.5 μg/mL CpG, 1.25 μg/mL cGAMP, 0.5 μg/mL MPLA, 0.5 μg/mL polyI:C, 0.5 μg/mL R848. PBS served as a negative control. The result indicates that combinations of CpG+cGAMP, CpG+MPLA, CpG+R848, and MPLA+R848 can synergistically stimulate TNF-α expression in BMDCs.

FIG. 4 displays scanning electron microscopy (SEM) images of porous silicon microparticles (Porous silicon μ-particle) and porous silica nanoparticles (Porous silica NP), and transmission electron microscopy (TEM) image of a lipid-based mRNA nanoparticle (Lipid-based mRNA NP). In the SEM image for porous silicon μ-particle, nanopores can be visualized. In the TEM for lipid-based mRNA NP, the dark mRNA core is surrounded with a light shell of lipids.

FIG. 5 displays confocal microscopy images of porous silicon μ-particles loaded with fluorescent dye-labeled CpG. Left panel shows particles under bright field, and right panel shows green fluorescent CpG in the μ-particles.

FIG. 6 is a high performance liquid chromatography (HPLC) elution profile showing separation of 2′3′-cGAMP, CpG, and a Her2 peptide that were used to prepare a μ-particle-based peptide vaccine (μGCHer2). All 3 substances were detected at 254 nm wavelength.

FIG. 7 shows cytokine expression levels in culture media of GM-CSF/IL-4-induced BMDCs after cells were co-incubated for 24 hours with μ-particle alone (μ-particle), cGAMP-loaded μ-particle (μG), CpG-loaded μ-particle (μC), or cGAMP and CpG-loaded μ-particle (μGC). PBS served as a negative control. ***: p<0.001. The results indicate potent stimulation of IFN-β and TNF-αexpression in cells treated with μGC.

FIG. 8 compares IFN-β and TNF-αexpression in GM-CSF/IL-4-induced BMDCs after cells were co-incubated for 24 hours with an equal amount of adjuvants (CpG and MPLA) packaged either in liposomes (Liposome) or in μ-particles (μ-particle). *: p<0.05. The result shows that adjuvants packaged in the μ-particle were more potent than in liposomes in stimulating cytokine expression, indicating that both soluble adjuvants (CpG and MPLA) and μ-particle are needed for maximum stimulation potential.

FIG. 9 shows activation and tumor infiltration of T cells after mice with B16 tumor were treated with a melanoma-specific vaccine comprised of μ-particle, tyrosinase-related protein 2 (Trp2)-specific antigen peptide, with or without CpG and cGAMP. μTrp2: μ-particle+Trp2 peptide, μG-Trp2: μ-particle+cGAMP+Trp2, μCTrp2: μ-particle+CpG+Trp2, μGC Trp2: μ-particle+cGAMP+CpG+Trp2. Panel a, Treatment schedule. Panel b, Analysis of IFN-γ-expressing splenocytes with an ELISpot assay. Panel c, Flow cytometry analysis of Trp2 antigen-specific T cells in the spleens after cells were stained with a Trp2-specific pentamer. Panel d, Histological analysis of CD3⁺ T cell infiltration into lung metastatic B16 tumor nodules. CD3⁺ T cells were stained in brown. PBS served as a negative control. **: p<0.01. The results indicate that μGCTrp2 treatment stimulated potent anti-tumor immune responses including significantly increased total number of IFN-γ-expressing cells and antigen-specific T cells in the spleen and tumor-infiltrated T cells in the lung.

FIG. 10 displays number of tumor nodules in the lungs after mice with lung metastatic melanoma were treated with μTrp2, μGCTrp2, Imject Alum (ThermoFisher) mixed with Trp2 peptide (AlumTrp2), or Alum mixed with cGAMP, CpG and Trp2 peptide (AlumGCTrp2). PBS served as a negative control. *: p<0.05; **: p<0.01. The result shows that μGC-based vaccine (μGCTrp2), but not Alum-based vaccine (AlumGCTrp2), was effective in eradicating tumor metastasis in the lung.

FIG. 11 shows survival curves after mice with lung metastatic B16 tumors were treated twice (on days 3 and 10) with individual vaccines. PBS served as a negative control. **: p<0.01; ***: p<0.001. The result shows that mice treated with μGC-based vaccine (μGCTrp2) had the biggest survival benefit.

FIG. 12 shows survival curves after mice with lung metastatic B16 tumors were treated either with μGCTrp2 or with a poly-ICLC-based vaccine (Poly-ICLC+Trp2). PBS served as a negative control. **: p<0.01. The result indicates that the μGC-based vaccine (μGCTrp2) was more potent than the poly-ICLC-based vaccine in anti-cancer activity.

FIG. 13 shows anti-tumor immune responses from particulate vaccines in mice with primary Her2-positive breast cancer. Particulate vaccines were prepared with cGAMP, CpG and a Her2-specific antigen peptide that were loaded into the μ-particle (μGCHer2). Panel a, Treatment schedule. Panel b, Histological analysis on CD3⁺ T cell infiltration into Her2-positive TUBO tumor. CD3⁺ T cells were stained in brown, and their levels both at the tumor boundary and inside the tumor were quantified and displayed. PBS served as a negative control. ***: p<0.001. The result indicates that μGCHer2 treatment effectively promoted tumor infiltration of T cells.

FIG. 14 shows inhibition of tumor growth after mice with primary TUBO tumors were treated twice on days 3 and 10 with μGCHer2 or LipoGCHer2. LipoGCHer2 was prepared by packaging cGAMP, CpG and a Her2-specific antigen peptide into liposomes, and μGCHer2 was prepared by loading LipoGCHer2 into the μ-particles. PBS served as a negative control. *: p<0.05; **: p<0.01; ***: p<0.001. The result indicates that the μGC-based vaccine (μGCHer2) was more potent in inhibiting breast cancer growth than the LipoGC vaccine (LipoGCHer2).

FIG. 15 shows inhibition of TUBO tumor growth after mice with primary TUBO tumors were treated twice on days 3 and 10 with PBS or a silica-based vaccine prepared by mixing cGAMP, CpG, and Her2-specific antigen peptide with porous silica particles (SiO₂+GCp66). ***: p<0.005. The result indicates that the silica-based vaccine (SiO₂+GCp66) was effective in promoting anti-tumor activity.

FIG. 16 shows anti-tumor activity from particulate vaccines on a mouse model of colon cancer. Particulate vaccines were prepared with a gp70 antigen peptide and μGC (μGCgp70) or a gp70 antigen peptide and poly-ICLC (polyICLC+gp70). Panel a, Histological analysis of CD3⁺ T cell infiltration into the CT26 colon cancer. CD3⁺ T cells were stained in brown. Panel b, Inhibition of CT26 tumor growth after tumor-bearing mice were treated with μGCgp70 or polyICLC+gp70 twice on days 3 and 10. PBS served as a negative control. *: p<0.05; **: p<0.01; ***: p<0.001. The result indicates that μGC-based vaccine was more effective than poly-ICLC-based vaccine in inhibiting tumor growth.

FIG. 17 shows plasma antibody levels in mice after they were treated twice (on days 0 and 13) with phosphate buffer saline (Mock), alum-based vaccine (Alum+RBD), or μGC-based vaccine (μGC+RBD). The antigen used in this study was a recombinant receptor-binding domain (RBD) of the SARS-CoV-2 Spike protein. The results indicate that vaccination with μGC+RBD triggered time-dependent increase in IgG1, IgG2a and IgG2b antibody levels, while treatment with Alum+RBD stimulated delayed IgG1 response only.

FIG. 18 shows protective efficacy from vaccines against SARS-CoV-2 Delta variant. Three groups of 6 to 8-week-old ACE2 transgenic mice were treated twice (on days 0 and 21) with phosphate buffer saline (Mock), Alum+RBD, or μGC+RBD. On day 35, all mice were treated intranasally with 1×10⁴ PFU SARS-CoV-2 Delta variant. Four days after viral challenge, lungs were collected, and SARS-CoV-2 viral titers in lung tissues were measured with plaque assay. The results indicate that vaccination with μGC+RBD protected mice from viral infection in the lung, while treatment with Alum+RBD only partially reduced viral load in the lungs.

FIG. 19 shows structure and composition of an mRNA vaccine particle (MVP) that is composed of a protamine/mRNA core (Core) and a lipid shell. The protamine/mRNA core is prepared by mixing positively charged protamine and negatively charged mRNA molecules. MVP is prepared by mixing the Core with 4 lipids (EDOPC, DOPE, cholesterol, and DSPE-PEG2k). A vehicle (Vehicle) prepared by mixing protamine and 4 lipids serves as a negative control for MVP. All 3 reagents (Core, Vehicle, and MVP) are used in studies to determine the proper adjuvant(s) for mRNA vaccine.

FIG. 20 shows IFN-β and TNF-αlevels in growth media of BMDCs after cells were treated with imiquimod (a TLR7 agonist), Vehicle, Core, or MVP2. ***: p<0.005; ****: p<0.001. The results indicate that imiquimod and MVP were same as potent in stimulating IFN-β expression, and part of the activity in MVP was from the mRNA-free Vehicle. The results also indicate that imiquimod and Vehicle were same as potent in stimulating TNF-α expression, and MVP was more potent than both of them in triggering TNF-α expression.

FIG. 21 shows IFN-β and TNF-α levels in growth media of BMDCs derived from wild-type mice (Wild-type), Sting knockout mice (Sting knockout), or Tlr7 knockout mice (Tlr7 knockout). Cells were treated with imiquimod, Vehicle, Core, or MVP.**: p<0.01; ns: not significant. The results indicate that STING signaling was essential for Vehicle- and MVP-stimulated IFN-βexpression, while TLR7 signaling was required for maximum MVP activity, but not for Vehicle-stimulated IFN-β expression. In contrast, neither STING nor TLR7 was required for Vehicle- or MVP-stimulated TNF-α expression.

FIG. 22 shows IFN-β levels in growth media of BMDCs derived from wild-type mice (Wild-type) or Sting knockout mice. Cells were treated with the STING agonist cGAMP, Vehicle (Vehicle with EDOPC), Vehicle prepared with DOTAP (Vehicle with DOTAP), MVP (MVP with EDOPC), or MVP prepared with DOTAP (MVP with DOTAP). *: p<0.05. The result indicates that EDOPC in Vehicle and MVP was essential for STING-dependent stimulation of IFN-βexpression. Replacing EDOPC with DOTAP in Vehicle or MVP wiped out stimulatory activity.

FIG. 23 shows tumor growth curves after mice with MC38 colon cancer or B16 melanoma were treated with vaccines. Both MC38 and B16 tumor cells were engineered to express an ovalbumin antigen (OVA). Tumor-bearing mice were treated with PBS control, Vehicle control, MVP prepared with GFP mRNA (as another control since GFP is not relevant to OVA), or MVP prepared with OVA mRNA on days 3 and 10. ***: p<0.005; ****: p<0.001. The results indicate that OVA MVP is very potent in inhibiting growth of OVA-expressing MC38 and B16 tumors in the respective murine models.

FIG. 24 compares antitumor activity from OVA MVP in murine model of B16 melanoma in wild-type and Sting knockout mice. ****: p<0.001; ns: not significant. The result shows there was no significant difference on tumor growth between wild-type (WT, PBS) and Sting knockout (Sting KO, PBS) mice after they were treated with PBS control. In the meanwhile, OVA MVP treatment completely inhibited tumor growth in wild-type mice (WT, OVA MVP), but only partially inhibited tumor growth in Sting knockout mice (Sting KO, OVA MVP), indicating STING signaling was needed for maximum MVP activity.

### DESCRIPTION OF THE PREFERRED EMBODIMENT

As used herein, the term “bone marrow-derived dendritic cell (BMDC)” refers to dendritic cells differentiated from bone marrow cells after co-incubation of bone marrow cells with GM-CSF and IL-4, or with Flt3 ligand.

As used herein, the term “adjuvant” refers to a Toll-like receptor ligand, a STING agonist, or any other compounds that promote cells to produce IFN-β, TNF-α, and other inflammatory cytokines.

As used herein, the term “adjuvant combination” refers to two or more adjuvants mixed together.

As used herein, the term “vaccine” refers to a formulation that consists of at least one adjuvant and one antigen or antigen source (such as an antigen-encoding mRNA).

As used herein, the term “particulate vaccine” refers to a vaccine that is packaged in the form of a nanoparticle or a microparticle.

The present invention provides a method to identify adjuvants or adjuvant combinations that can be used for vaccine development. A desired adjuvant is able to potently stimulate antigen-presenting cells to produce type I interferons (IFN-α and IFN-β) and/or other inflammatory cytokines including, but not limited to, TNF-α. Such cytokines will not only promote maturation of the antigen-presenting cells, but also modify the local microenvironment to facilitate antigen presentation and T cell activation.

The present invention also provides a method to identify adjuvants and their combinations that further enhance activity from particulate vaccines. Vaccines are commonly packaged in the form of nanoparticles and microparticles. The building blocks of certain particulate vaccines pose adjuvant activity. For an example, the porous silicon-based μ-particle can moderately activate TRIF/MAVS-mediated signal transduction pathways, leading to IFN-α/β expression in dendritic cells (10). It has also been reported that mRNA nanoparticles have the potential to stimulate TLR7/8 signaling (11). It is desirable to identify inorganic or organic adjuvant molecules that work together with nanoparticles or microparticles to synergize activation of signal transduction pathways leading to secretion of inflammatory cytokines in antigen-presenting cells.

In addition, the present invention provides compositions of adjuvants and adjuvant combinations that constitute an essential part of a vaccine. Such adjuvants and adjuvant combinations are applied to prepare vaccines to treat diseases in humans and vertebrate animals, including cancer and infectious diseases.

### EXAMPLES

The present invention is more particularly described in the following non-limiting examples, which are intended to be illustrative only, as numerous modifications and variations therein will be apparent to those skilled in the art.

### Example 1

### Generation and Characterization of BMDCs

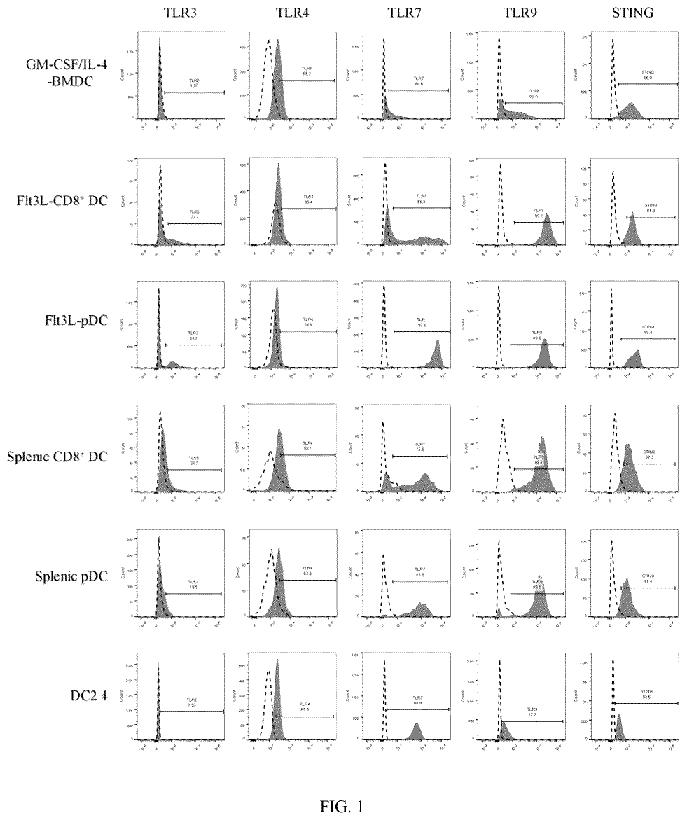

BMDCs were generated by co-incubation of bone marrow cells with GM-CSF/IL-4 or Flt3 ligand. To generate GM-CSF/IL-4-induced BMDCS, bone marrow cells were flushed out from mouse femur and tibia with 2% fetal bovine serum (FBS)-containing phosphate buffer saline (PBS). After removal of red blood cells, bone marrow cells were grown in a 37° C. incubator with 5% CO₂ in RPMI-1640 supplemented with 20 ng/ml recombinant murine GM-CSF and IL-4 for 6 days. Cell culture medium was refreshed every other day. To induce BMDC with Flt3 ligand, bone marrow cell culture was supplemented with 200 ng/mL Flt3 ligand. Cell culture medium was refreshed on day 5, and continued for another 5 days. CD8⁺DCs and B220⁺pDCs were isolated from Flt3L-induced BMDCs with a CD8⁺ DC isolation kit (Miltenyi) and with B220 microbeads (Miltenyi). To characterize BMDCs, cells were stained with anti-CD40, anti-CD80 or anti-CD86 antibody to determine maturation status and with anti-TLR and anti-STING antibodies to determine protein expression. Flow cytometry was applied in cell characterization (FIG. 1).

### Example 2

### Stimulation of Cytokine Expression by Soluble Adjuvants in BMDCs

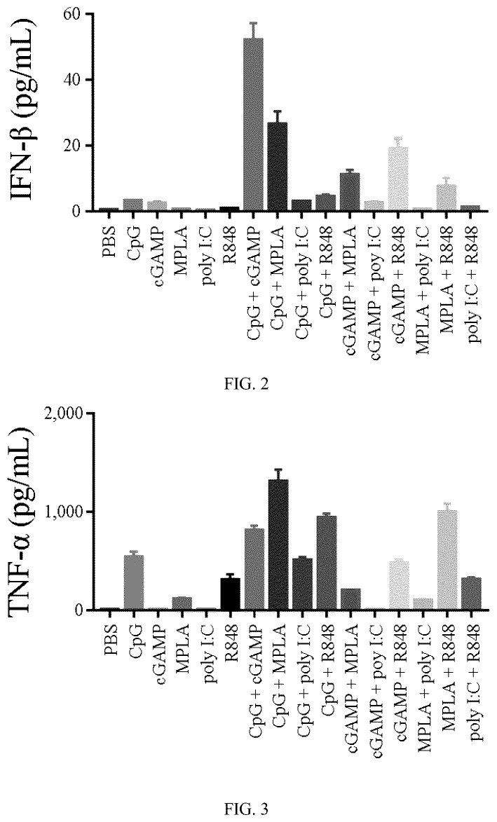

GM-CSF/IL4-induced BMDCs were seeded into a 24-well plate with a seeding density of 5×10⁵ cells/well, and treated with the following reagents either as a single agent or in combination: 2.5 μg/mL CpG, 1.25 μg/mL cGAMP, 0.5 μg/mL MPLA, 0.5 μg/mL polyI:C, 0.5 μg/mL R848. Cell growth media were collected 24 hours later, and IFN-β and TNF-αlevels were measured with ELISA kits (FIG. 2, FIG. 3).

### Example 3

### Nanoparticles and Microparticles as Carriers for Vaccines

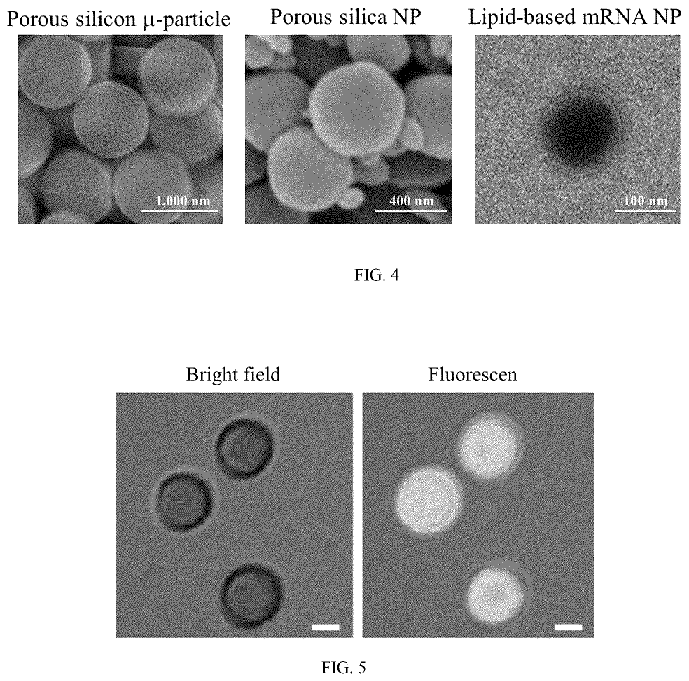

The μ-particles were produced by a combination of photolithography and electrochemical etching, and their surface was conjugated with (3-aminopropyl)triethoxysilane(15). Porous silica nanoparticles were chemically synthesized. Liposomes encapsulated with mRNA molecules were prepared with a microfluidic device. All particles have been characterized based on their size, shape, and surface chemistry, including with SEM or TEM imaging (FIG. 4).

### Example 4

### Preparation of Vaccine Particles with μ-Particle

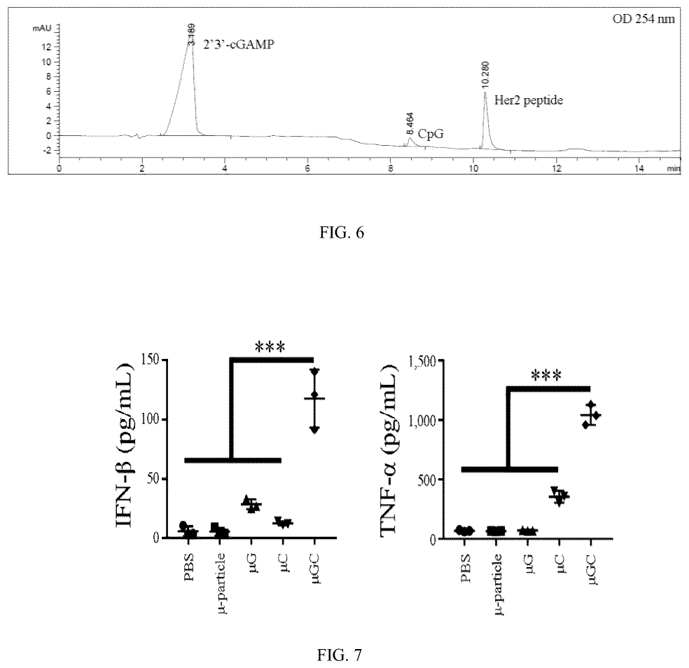

Soluble adjuvants and antigens were dissolved in water, and mixed with 20 mg/ml 1,2-dioleoyl-sn-glycero-3-phosphocholine, t-butanol and 0.1% Tween-20. The sample was then freeze-dried in a lyophilizer. Liposomes were reconstituted by adding water into the powder, and were then loaded into μ-particles by brief sonication. Effective loading of fluorescently-labeled adjuvants into the μ-particle can be confirmed under the fluorescent microscopy (FIG. 5). The complete μGC-based vaccine contains 10 μg CpG, 5 μg cGAMP and 100 μg antigen peptide (the p66 Her2 antigen peptide, gp70 antigen peptide, or Trp2 antigen peptide) in 0.6 billion 1 μm-size particles. Individual components in the vaccine can be measured with HPLC (FIG. 6).

### Example 5

### Stimulation of Cytokine Expression by Particulate Vaccines in BMDCs

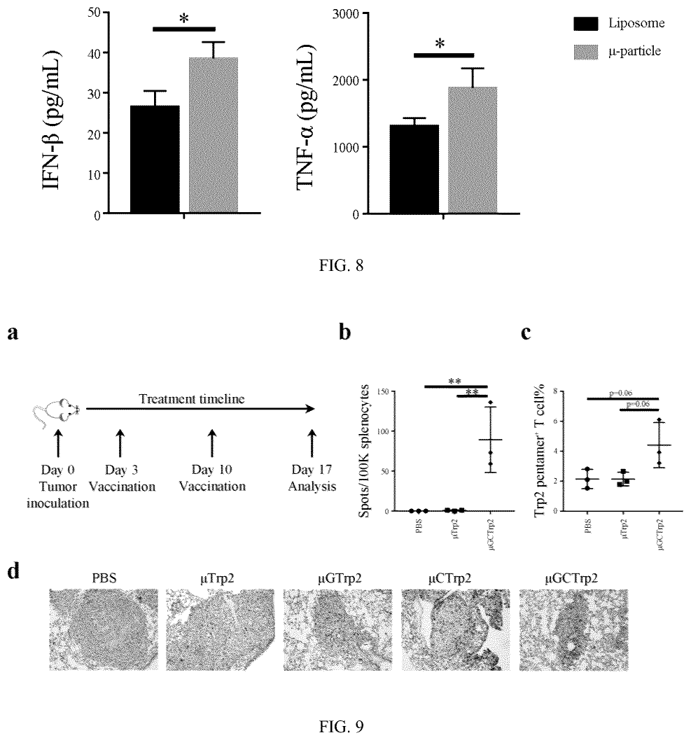

GM-CSF/IL4-induced BMDCs were seeded into 24-well plates at a seeding density of 5×10⁵ cells/well, and treated with 1_1-particle-based vaccines. Cell growth media were collected 24 hours later, and levels of IFN-β and TNF-α were measured with ELISA kits (FIG. 7). In a separate study, BMDCs were co-incubated with a liposomal vaccine or a μ-particle-based vaccine, and cytokine levels in growth media were determined (FIG. 8).

### Example 6

### Measurement of T Cell Activation

To study T cell activation ex vivo, C57BL6 mice were inoculated with B16 melanoma (on day 0) by tail vein injection, and treated twice (on days 3 and 10) with partial or complete vaccines containing 100 μg Trp2 peptide in the foot pads. Mice were euthanized 7 days after the second vaccination (on day 17), and spleens were collected to process for single cell isolation (FIG. 9, panel a). ELISpot assay was applied to determine antigen-specific T cell activity. Briefly, splenocytes were seeded at 1×10⁵ cell/well in an anti-IFN-γ-coated MultiScreen-IP plate (Millipore), and stimulated with 10 μg/mL Trp2 peptides in growth medium for 36 hours. The plate was then washed and incubated with biotinylated anti-mouse IFN-γ antibody, followed by staining with an avidin-HRP (FIG. 9, panelb). Splenocytes were stained with Trp2 pentamer, and flow cytometry was applied to measure pentamer-positive T cells (FIG. 9, panel c). In the meanwhile, lungs with B16 tumor nodules were processed and stained with anti-CD3 antibody to determine tumor-infiltrated T cells (FIG. 9, panel d).

### Example 7

### Evaluation of Anti-Tumor Activity in Mice with Melanoma

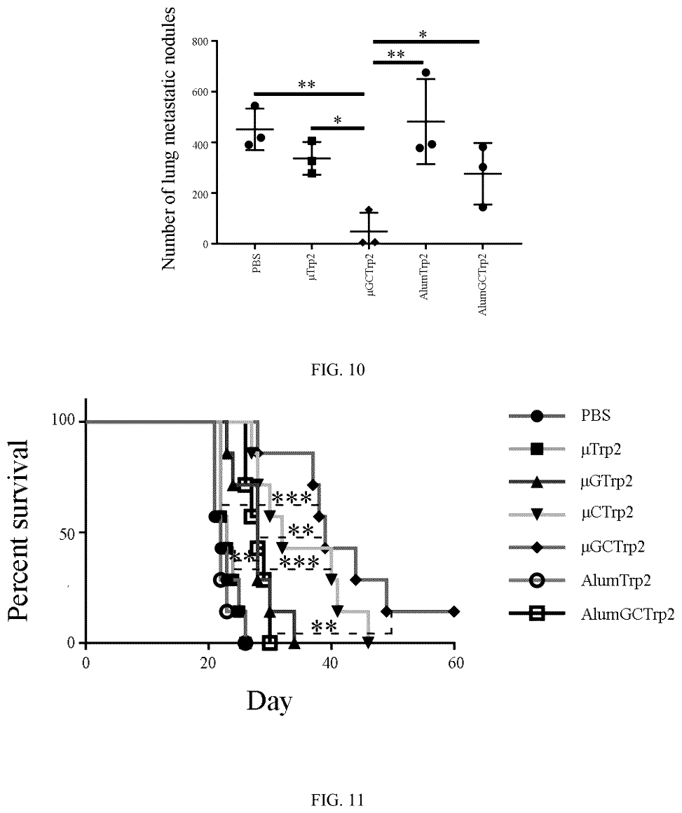

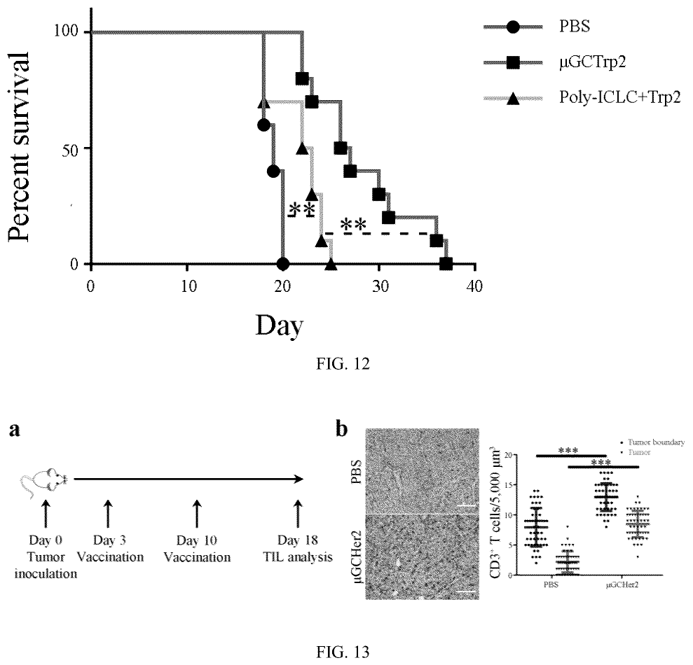

Murine model of lung metastatic melanoma was generated by inoculating murine B16 melanoma cells at 2.5×10⁵ cells/mouse by tail vein injection into 6 to 8-week-old C57BL6 mice. Three days after tumor inoculation, mice were randomly allocated into treatment groups, and treated with partial or complete vaccines prepared with a Trp2 antigen peptide. They were boosted with the same vaccine one week after the first treatment. Mice were euthanized 5 days after the second treatment, and number of black metastatic tumor nodules in the lung was counted (FIG. 10). In a separate study, mice with lung metastatic B16 melanoma were treated twice (on days 3 and 10) with partial or complete vaccines. They were euthanized when one of the endpoints is met including lethargic, hunched back, ruffled fur, and loss of 15% body weight. Kaplan-Meier plots were generated based on animal survival, and their survival benefits were compared (FIG. 11). In another study, anti-tumor efficacy was compared after mice with lung metastatic B16 melanoma were treated twice (on days 3 and 10) with μGCHer2 or a polyICLC-based vaccine. Kaplan-Meier plots were generated, and survival benefit was compared (FIG. 12).

### Example 8

### Evaluation of Anti-Tumor Activity in Mice with Primary Breast Cancer

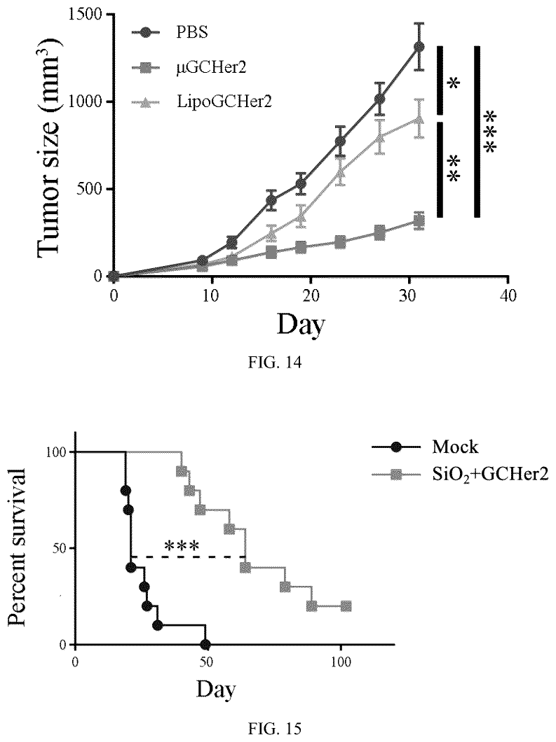

Murine model of primary breast cancer was generated by inoculating Her2-expressing TUBO tumor cells in the mammary gland fat pads of 6 to 8-week-old female BALB/c mice at 1×10⁶ cells/mouse. Mice were treated with PBS control or μGCHer2 vaccine prepared with a Her2 antigen peptide in the fat pads once three days after tumor inoculation and the second time one week after the first vaccination. Mice were euthanized 3 days later, and tumor samples were harvested and processed to stain with an anti-CD3 antibody. Number of tumor-infiltrated T cells were compared in the control and μGCHer2 vaccination groups (FIG. 13). To test therapeutic efficacy from Her2-specific vaccines, BALB/c mice with Her2-expressing TUBO tumors were treated with a LipoGCHer2 or μGCHer2 vaccine in the fat pads once three days after tumor inoculation and the second time one week after the first vaccination. Tumor growth was monitored on daily basis, and tumor growth curves were generated and compared (FIG. 14).

### Example 9

### Evaluation of Anti-Tumor Activity in Mice with Metastatic Breast Cancer

To test anti-tumor immune responses from silica-based vaccines, BALB/c mice with metastatic TUBO breast tumors (generated by intracardiac injection of TUBO tumor cells) were treated twice by intradermal inoculation with PBS control (Mock) or a vaccine prepared with porous silica nanoparticle (SiO₂+GCHer2). Mice were monitored on daily basis, and euthanized when they showed signs of terminal illness. Kaplan-Meier plots were generated based on animal survival, and survival benefit was compared (FIG. 15).

### Example 10

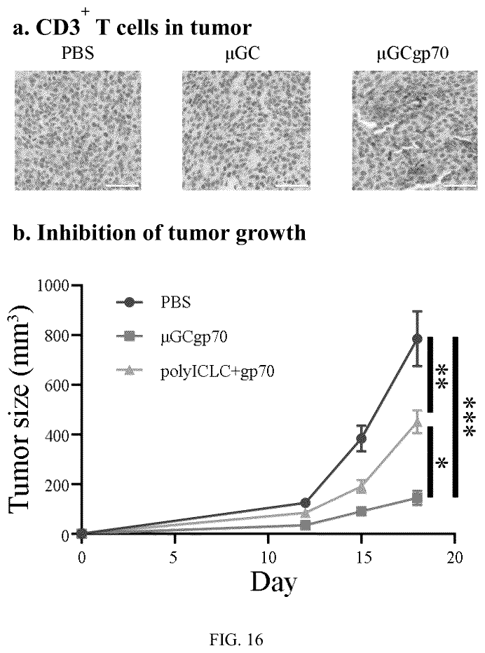

Evaluation of anti-tumor activity in mice with colon cancer Murine model of colorectal cancer was generated by inoculating CT26 tumor cells subcutaneously into 6 to 8-week-old BALB/c mice. Mice with CT26 tumors were treated twice (on days 3 and 10) with PBS control, μGC control, or μGCgp70 vaccine prepared with a gp70 antigen peptide. Mice were euthanized 3 days after the second vaccination, and tumor samples were processed for T cell staining with an anti-CD3 antibody (FIG. 16, panel a). In a separate study, BALB/c mice with CT26 colon tumor were treated twice (on days 3 and 10) by intradermal inoculation with PBS control, μGCgp70, or polyICLC+gp70. Tumor growth was monitored on daily basis, and tumor growth curves were generated and compared (FIG. 16, panel b).

### Example 11

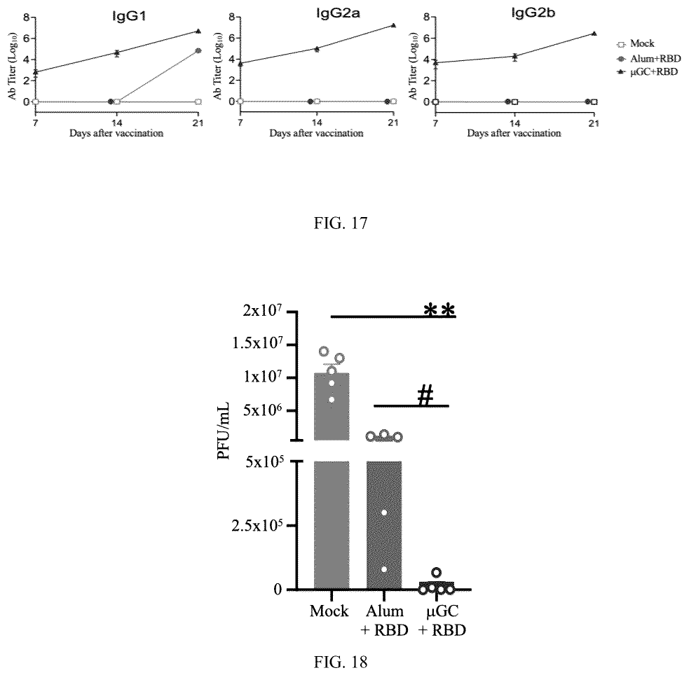

Evaluation of humoral responses from particulate vaccines against COVID-19 Two vaccines were prepared using a recombinant receptor-binding domain (RBD) of the COVID-19 Spike protein. μGC+RBD was prepared by loading liposomal GC+RBD (containing 1 μg CpG, 0.5 μg cGAMP, and 25 μg RBD) into 60 million μ-particles. Alum+RBD was prepared by mixing 25 μg RBD with 25 μL Imject Alum (ThermoFisher). To test humoral responses from the above vaccines, 6 to 8-week-old BALB/c mice were treated intradermally with PBS control (Mock), Alum+RBD, or μGC+RBD on days 0 and 13, and blood samples were collected on days 7, 14 and 21. ELISA assays were performed to measure plasma IgG1, IgG2a and IgG2b levels, and time-dependent antibody titer changes were plotted (FIG. 17).

### Example 12

### Evaluation of Anti-COVID-19 Activity from Vaccines

Three groups of 6 to 8-week-old ACE2 transgenic mice were immunized twice (on days 0 and 21) with Mock (PBS), Alum+RBD, or μGC+RBD. On day 35 post first vaccination, all mice were challenged intranasally with 1×10⁴ plaque-forming unit (PFU) SARS-CoV-2 Delta variant. Mice were euthanized 4 days later, and lungs were collected and processed to measure viral load by plaque assay. Results were presented as number of PFU. Lack of plaque formation indicates that all viruses have been cleaned from the lungs indicating potent protection from viral infection (FIG. 18).

### Example 13

### Preparation of mRNA Vaccine Particles (MVP)

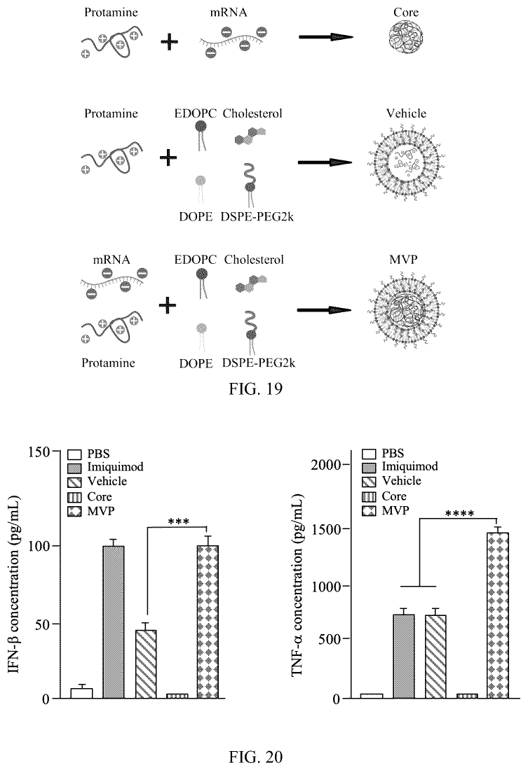

mRNA vaccine contains an mRNA core and a lipid shell. To prepare the mRNA core, an mRNA solution was mixed with a protamine sulfate solution at 1:1 (weight ratio) in a NanoAssemblrbenchtop microfluidic instrument (Precision NanoSystems). To prepare the organic phase, 1,2-dioleoyl-sn-glycero-3-ethylphosphocholine (EDOPC, 20 mg/mL), 1,2-dioleoyl-snglycero-3-phosphatidyl-ethanolamine (DOPE, 20 mg/mL), cholesterol (10 mg/mL), and 1,2-distearoyl-sn-glycero-3-phosphoethanolamine-N-amino (polyethylene glycol)-2000 (DSPE-PEG2k, 2 mg/mL) were dissolved in ethanol and mixed at 34:30:35:1 (molar ratio). To prepare mRNA vaccine particle (MVP), the aqueous mRNA core was mixed with the organic solution in the NanoAssemblr benchtop microfluidic instrument. To prepare mRNA-free vehicle, an aqueous phase containing protamine only was mixed with the organic solution in the NanoAssemblr benchtop microfluidic instrument (FIG. 19).

### Example 14

### Stimulation of Cytokine Production by mRNA Vaccine

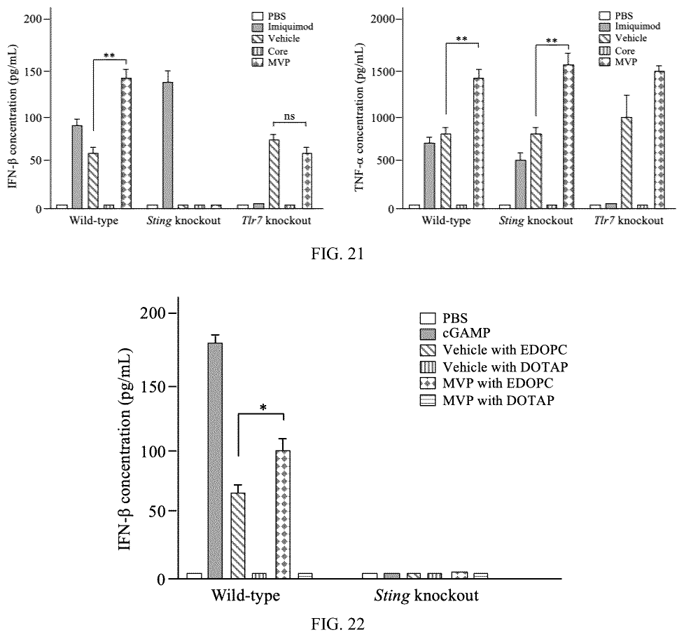

GM-CSF/IL4-induced BMDCs were seeded into 24-well plates at a seeding density of 5×10⁵ cells/well, and treated with PBS control, the TLR7 agonist imiquimod, mRNA-free vehicle control, mRNA core control, or mRNA vaccine (MVP) for 24 hours. Cell growth media were collected and IFN-b and TNF-α levels were measured with ELISA assay (FIG. 20). To determine pathways that play important roles on stimulation of cytokine production, BMDCs were induced from bone marrow cells collected from wild-type mice, Sting knockout mice, and Tlr7 knockout mice. Cells were treated with PBS control, mRNA-free vehicle control, mRNA core control, or mRNA vaccine (MVP) for 24 hours, and cell growth media were collected 24 hours later for measurement of cytokine levels. Lack or dramatically reduced cytokine expression in BMDCs derived from gene knockout mice (comparing to those from wild-type mice) indicates the importance of the gene-of-interest in mediating mRNA vaccine-stimulated cytokine expression (FIG. 21).

### Example 15

### Identification of Key Adjuvant Component(s) in mRNA Vaccine

Individual components in mRNA vaccine were swapped with other reagents in order to identify key molecule(s) responsible for stimulation of cytokine expression (and hence adjuvant activity). In order to determine the role of the charged lipid (i.e., EDOPC), positively charged 1,2-dioleoyl-3-trimethylammonium-propane (DOTAP) was used to replace EDOPC to prepare vehicle and mRNA vaccine. The resulting vehicle (Vehicle with DOTAP) and mRNA vaccine (MVP with DOTAP) were applied to compare with the parental vehicle (Vehicle with EDOPC) and mRNA vaccine (MVP with EDOPC) on stimulation of cytokine expression after BMDC treatment. Lack or dramatically reduced cytokine expression in BMDCs after treatment with the new vaccine particle (compared to the parental vaccine particle) indicates the importance of the molecule-of-interest in mediating mRNA vaccine-stimulated cytokine expression (FIG. 22).

### Example 16

### Anti-Tumor Activity from mRNA Vaccine

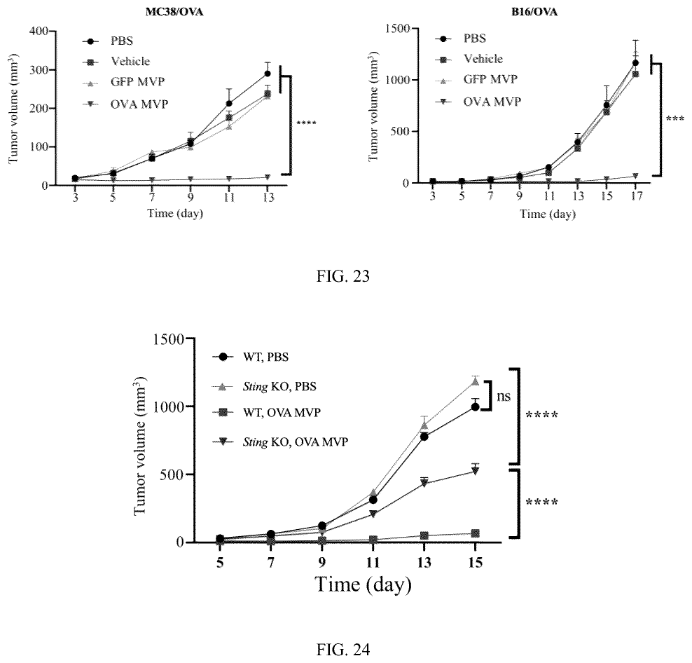

MC38 colon cancer cells and B16 melanoma cells were engineered with ovalbumin expression. The resulting cells, MC38/OVA and B16/OVA, were applied to generate murine models of colorectal cancer and melanoma by inoculating subcutaneously in C57BL6 mice. Mice were treated twice (on days 3 and 10) with PBS control (PBS), mRNA-free vehicle control (Vehicle), mRNA vaccine prepared with mRNA encoding GFP which is not related to ovalbumin (GFP MVP), or mRNA vaccine prepared with mRNA encoding ovalbumin (OVA MVP). Tumor growth was monitored on daily basis, and time-dependent tumor growth curves were generated (FIG. 23). In a separate study, B16/OVA cells were inoculated subcutaneously into wild-type (WT) and Sting knockout (Sting KO) mice. Mice were treated twice (on days 3 and 10) either with PBS control or with OVA MVP. Tumor growth was monitored on daily basis, and time-dependent tumor growth curves were generated (FIG. 24).

All patents and publications mentioned in the specification of the invention indicate that these are public technologies in the field, which is used by the invention. All patents and publications quoted herein are also listed in the references, as each publication is specifically referenced separately. The invention described herein may be implemented in the absence of any one or more elements, one or more restrictions, which are not specially specified herein. For example, the terms “including”, “comprising” and “consisting of” in each embodiment is replaced by the other two. The so-called “one” herein only means “one”, while excluding or only does not mean only including one, it also means including more than two. The terms and expressions used here are described without limitation, and it is not intended herein to indicate that the terms and interpretations described in this document exclude any equivalent feature, but it is understood that any appropriate alteration or modification may be made to the extent of the invention and claims. It is understood that the embodiments described in the present invention are some preferred exemplary embodiments and features. Any person skilled in the art makes some variations and changes based on the essence described in the present invention. These variations and changes are also considered within the scope of the invention and the scope limited by the independent claims and the dependent claims.

### REFERENCES

1. Woo S R, Corrales L, Gajewski T F. Innate immune recognition of cancer. Annu Rev Immunol. 2015; 33:445-74. Epub 2015/01/27. doi: 10.1146/annurev-immunol-032414-112043. PubMed PMID: 25622193.
2. Broz M L, Krummel M F. The emerging understanding of myeloid cells as partners and targets in tumor rejection. Cancer Immunol Res. 2015; 3(4):313-9. doi: 10.1158/2326-6066.CIR-15-0041. PubMed PMID: 25847968; PMCID: 4391275.
3. Iwasaki A, Omer S B. Why and How Vaccines Work. Cell. 2020; 183(2):290-5. doi: 10.1016/j.cell.2020.09.040. PubMed PMID: WOS:000578604900002.
4. Chen D S, Mellman I. Oncology meets immunology: the cancer-immunity cycle. Immunity. 2013; 39(1):1-10. Epub 2013/07/31. doi: S1074-7613(13) 00296-3 [pii] 10.1016/j.immuni.2013.07.012. PubMed PMID: 23890059.
5. Smyth M J, Ngiow S F, Ribas A, Teng M W. Combination cancer immunotherapies tailored to the tumour microenvironment. Nat Rev Clin Oncol. 2016; 13(3):143-58. Epub 2015/11/26. doi: 10.1038/nrclinonc.2015.209 nrclinonc.2015.209 [pii]. PubMed PMID: 26598942.
6. Sagiv-Barfi I, Czerwinski D K, Levy S, Alam I S, Mayer A T, Gambhir S S, Levy R. Eradication of spontaneous malignancy by local immunotherapy. Sci Transl Med. 2018; 10(426):aan4488. Epub 2018/02/02. doi: 10.1126/scitranslmed.aan4488. PubMed PMID: 29386357; PMCID: PMC5997264.
7. Corrales L, Glickman L H, McWhirter S M, Kanne D B, Sivick K E, Katibah G E, Woo S R, Lemmens E, Banda T, Leong J J, Metchette K, Dubensky T W, Jr., Gajewski T F. Direct Activation of STING in the Tumor Microenvironment Leads to Potent and Systemic Tumor Regression and Immunity. Cell Rep. 2015; 11(7):1018-30. Epub 2015/05/12. doi: 10.1016/j.celrep.2015.04.031. PubMed PMID: 25959818; PMCID: PMC4440852.
8. Akira S. Innate immunity and adjuvants. Philosophical transactions of the Royal Society of London Series B, Biological sciences. 2011; 366(1579):2748-55. doi: 10.1098/rstb.2011.0106. PubMed PMID: 21893536; PMCID: 3146784.
9. Schlom J. Therapeutic cancer vaccines: current status and moving forward. J Natl Cancer Inst. 2012; 104(8):599-613. Epub 2012/03/08. doi: djs033 [pii] 10.1093/jnci/djs033. PubMed PMID: 22395641; PMCID: 3328421.
10. Xia X, Mai J, Xu R, Perez J E, Guevara M L, Shen Q, Mu C, Tung H Y, Corry D B, Evans S E, Liu X, Ferrari M, Zhang Z, Li X C, Wang R F, Shen H. Porous Silicon Microparticle Potentiates Anti-Tumor Immunity by Enhancing Cross-Presentation and Inducing Type I Interferon Response. Cell Rep. 2015; 11:957-66. Epub 2015/05/06. doi: S2211-1247(15) 00384-8 [pii] 10.1016/j.celrep.2015.04.009. PubMed PMID: 25937283.
11. Persano S, Guevara M L, Li Z, Ferrari M, Pompa P P, Shen H. Lipopolyplex potentiates anti-tumor immunity of mRNA-based vaccination. Biomaterials. 2017; 125:81-9.
12. Marichal T, Ohata K, Bedoret D, Mesnil C, Sabatel C, Kobiyama K, Lekeux P, Coban C, Akira S, Ishii K J, Bureau F, Desmet C J. DNA released from dying host cells mediates aluminum adjuvant activity. Nat Med. 2011; 17(8):996-1002. Epub 2011/07/19. doi: 10.1038/nm.2403 nm.2403 [pii]. PubMed PMID: 21765404.
13. Hornung V, Bauernfeind F, Halle A, Samstad E O, Kono H, Rock K L, Fitzgerald K A, Latz E. Silica crystals and aluminum salts activate the NALP3 inflammasome through phagosomal destabilization. Nat Immunol. 2008; 9(8):847-56. doi: 10.1038/ni.1631. PubMed PMID: 18604214; PMCID: PMC2834784.
14. Lindblad E B. Aluminium compounds for use in vaccines. Immunol Cell Biol. 2004; 82(5):497-505. Epub 2004/10/14. doi: ICB1286 [pii] 10.1111/j.0818-9641.2004.01286.x. PubMed PMID: 15479435.
15. Xu R, Zhang G, Mai J, Deng X, Segura-Ibarra V, Wu S, Shen J, Liu H, Hu Z, Chen L, Huang Y, Koay E, Huang Y, Liu J, Ensor J E, Blanco E, Liu X, Ferrari M, Shen H. An injectable nanoparticle generator enhances delivery of cancer therapeutics. Nat Biotechnol. 2016; 34(4):414-8. doi: 10.1038/nbt.3506. PubMed PMID: 26974511.

1. A method for identification of an adjuvant and adjuvant combination, comprising:
using an adjuvant or adjuvant combinations to treat at least one type of antigen-presenting cells and measuring amount of at least one cytokine produced by the at least one type of antigen-presenting cells.

2. The method according to claim 1, wherein the adjuvant comprises a hydrophilic or/and a hydrophobic molecule.

3. The method according to claim 1, wherein at least one cytokine has the property to stimulate at least one type of antigen-presenting cells.

4. The method according to claim 1, wherein the antigen-presenting cell is a dendritic cell, a macrophage, or a B lymphocyte.

5. The method according to claim 4, wherein the dendritic cell is derived from bone marrow cells.

6. The method according to claim 4, wherein the dendritic cell is isolated from peripheral blood or a tissue.

7. The method according to claim 4, wherein the dendritic cell is an immortalized cell line.

8. The method according to claim 1, wherein the hydrophilic or hydrophobic molecule is capable of stimulating expression of a type I interferon or an inflammatory cytokine.

9. The method according to claim 8, wherein the hydrophilic or hydrophobic molecule is a Toll-like receptor ligand or agonist.

10. The method according to claim 8, wherein the hydrophilic or hydrophobic molecule is a STING agonist.

11. The method according to claim 8, wherein the hydrophilic or hydrophobic molecule is a nucleotide analogue.

12. The method according to claim 8, wherein the hydrophilic or hydrophobic molecule is selected from a compound library based on its cytokine-stimulating property.

13. The method according to claim 8, wherein the hydrophilic or hydrophobic molecule is an mRNA molecule.

14. The method according to claim 1, wherein the cytokine can stimulate maturation of the antigen-presenting cells.

15. The method according to claim 14, wherein the cytokine is interferon-beta.

16. The method according to claim 14, wherein the cytokine is tumor necrosis factor-alpha.

17. The method according to claim 1, wherein the hydrophilic or hydrophobic molecule can be packaged into a nanometer-size or micrometer-size particle.

18. The method according to claim 17 wherein the particle is a porous silicon particle, a porous silica particle, or a lipid nanoparticle.

19. The method according to claim 17, wherein the hydrophilic or hydrophobic molecule packaged in a particle can stimulate cytokine expression in antigen-presenting cells.

20. The method according to claim 17, wherein the hydrophilic or hydrophobic molecule packaged in a particle has an equal or greater activity in stimulating cytokine expression in antigen-presenting cells compared to its free form.

21. The method according to claim 17 wherein the hydrophilic or hydrophobic molecule synergizes with a particulate component in stimulating cytokine expression in antigen-presenting cells.

22. The method according to claim 17, wherein the hydrophilic or hydrophobic molecule in the particle has the capacity to promote antigen processing and presentation in antigen-presenting cells.

23-50. (canceled)
